(19) United States
(12) Patent Application Publication
(10) Pub. No.: US 2023/0203017 A1
(43) Pub. Date: Jun. 29, 2023

(54) Protein Kinase C Beta Inhibitors and Uses Thereof

(71) Applicant: Kamal D. Mehta, Dublin, OH (US)

(72) Inventor: Kamal D. Mehta, Dublin, OH (US)

(21) Appl. No.: 17/563,351

(22) Filed: Dec. 28, 2021

(51) Int. Cl. C07D 403/14; C07D 498/22; A61P 3/04; A61P 35/00

(52) U.S. Cl. CPC C07D 403/14; C07D 498/22; A61P 3/04; A61P 35/00

(57) ABSTRACT

Provided herein are protein kinase Cβ inhibitors or pharmaceutical compositions thereof, for example, derivatives or analogs of bisindolylmaleimide with the general structure:

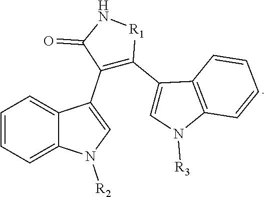

Also provided are methods for treating a metabolic disease, for example, obesity and obesity-related diseases in a subject by administering one or more times at least one of the protein kinase Cβ inhibitors or a pharmaceutical composition thereof.

### BACKGROUND OF THE INVENTION

### Field of the Invention

The present invention relates to the field of pharmaceutical compounds and therapeutic inhibitory compounds for treating a disease. More particularly, the present invention relates to protein kinase Cb (PKCb) inhibitors for treating obesity and related metabolic syndromes and disorders.

### Description of the Related Art

The prevalence of obesity and related disorders has been increasing all over the world (1, 2). Obesity is one of the greatest public health threats of the 21st century both due to health care costs as well as associated complications such as cardiovascular disease, liver disease, and type II diabetes (3-5). Obesity is accompanied by a concomitant rise in the prevalence of non-alcoholic fatty liver disease (NAFLD). Despite extensive efforts in the field, treatment modalities for obesity have been met with limited success.

The onset of obesity is a complex process that involves genetic and environmental factors (6, 7). It is widely recognized that lifestyle factors, such as excessive consumption of dietary fat and limited physical activity, promotes adiposity. Fats, mainly in the form of di- and triglycerides, contribute over 40% of the caloric content of western diet.

A hallmark of obesity is excessive expansion of body fat that is attributable to energy intake exceeding energy expenditure, creating a state of positive energy balance. Understanding of the mechanisms by which body acts to achieve and maintain energy balance is incomplete, but the emerging evidence supports existence of complex inter-organ networks that are needed to coordinate energy homeostasis (8, 9). Lipid overloads result in lipid redistribution among metabolic organs such as liver, adipose, and muscle and the interplay between these organs is important to maintain lipid homeostasis. The inter-organ communications are largely orchestrated by secreted biologically active molecules that modulate metabolic processes in target tissues via autocrine, paracrine, or endocrine mechanisms to modulate calorie storage and energy expenditure to regulate adiposity (10-14). Available research indicates that adiposity-induced dysfunctions within these cross-talks can lead to imbalances in energy metabolism and contribute to the pathogenesis of metabolic diseases (9).

Protein kinase C beta (PKC(3), a member of the serine/threonine kinase PKC family, regulates a wide range of cellular functions including nutrient metabolism and energy homeostasis (15). PKC is a lipid activatable enzyme that is known for its regulation by insulin. Global inactivation of PKCβ in normal mice or leptin-deficient (ob/ob mice) has beneficial effects on metabolism and protects from diet-induced adiposity, hepatic steatosis, and insulin-resistance (16-21). Cre/loxP mice lacking hepatic PKCβ were observed to be protected from diet-induced hepatic steatosis when subject to chronic fat feeding stress (20).

Obesity is a leading cause of morbidity and mortality worldwide. This epidemic has increased the demand for novel therapeutics targeted toward modulating appetite and/or energy metabolism. The search for clinically useful drugs has thus far met with limited success. Hence, identifying novel targets for preventing and treating obesity is crucial for management of this disease.

Thus, there is a need in the art for compounds that have a beneficial impact on lipid homeostasis and, therefore, obesity and related diseases. Particularly, the art is deficient in protein kinase Cβ inhibitors to treat these conditions. The present invention fulfills this longstanding need and desire in the art.

### SUMMARY OF THE INVENTION

The present invention is directed to a protein kinase Cβ inhibitor. The protein kinase Cβ inhibitor has a chemical structure

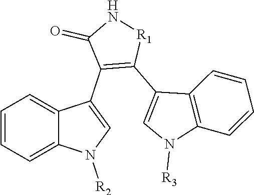

or a pharmaceutically acceptable salt thereof. In the structure R₁ is C═O or N and R₂ and R₃ independently are H, CH₃, or (CH₂)₄C≡N.

The present invention also is directed to a pharmaceutical composition. The pharmaceutical composition comprises at least one protein kinase Cβ inhibitor, as described herein, and a pharmaceutically acceptable carrier. The present invention is directed to a related pharmaceutical composition in which the at least one protein kinase Cβ further comprises a bisindolylmaleimide analog where R₁ and R₂ together with the indolyl nitrogens form a (dimethylamino)methyl-oxa-triazahexacyclic ring or a pharmaceutically acceptable salt thereof.

The present invention is directed further to a method for treating obesity and an obesity-related liver disease in a subject in need of such treatment. In the method a therapeutically effective dose of the pharmaceutical composition described herein is administered one or more times to the subject.

The present invention is directed further still to a method for treating a subject suffering from a metabolic disease. In the method, in a pharmaceutically acceptable carrier, a therapeutically effective dose of at least one protein kinase Cβ inhibitor with a chemical structure

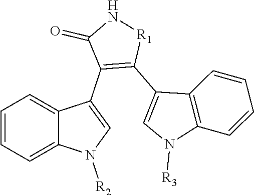

or a pharmaceutically acceptable salt thereof is administered at least once to the subject. In the structure R₁ is C═O or N, R₂ and R₃ independently are H, CH₃, or (CH₂)₄C≡N or R₁ and R₂ together with the indolyl nitrogens form a (dimethylamino)methyl-oxa-triazahexacyclic ring.

The present invention is directed further still to a related method for treating at least one of obesity or other obesity-related disease in a subject in need thereof. In the method, in a pharmaceutically acceptable carrier, a therapeutically effective dose of at least one protein kinase Cβ inhibitor with a chemical structure

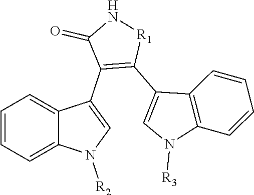

or a pharmaceutically acceptable salt thereof is administered, parenterally, at least once to the subject. In the structure R₁ is C═O or N, R₂ and R₃ independently are H, CH₃, or (CH₂)₄C≡N or R₁ and R₂ together with the indolyl nitrogens form a (dimethylamino)methyl-oxa-triazahexacyclic ring.

Other and further aspects, features, benefits, and advantages of the present invention will be apparent from the following description of the presently preferred embodiments of the invention given for the purpose of disclosure.

### BRIEF DESCRIPTION OF THE FIGURES

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1D shows steps to generation and phenotyping of mice with hepatocyte-specific PKCβ deficiency. FIG. 1A is a schematic of the targeting strategy used to generate PKCβ ^{fl/fl} and PKCβ^{Hep-/-} mice. FIG. 1B shows validation of effective DNA recombination by PCR analysis of genomic DNA. FIG. 1C shows western blot analysis of PKCβ protein expression in hepatocytes of PKCβ^{fl/fl}, PKCβ^{Hep+/-} and PKCβ^{Hep-/-} mice. FIG. 1D shows relative PKCβ protein levels in liver, white adipose tissue (WAT), muscle and brain of PKCβ^{fl/fl} and PKCβ^{Hep-/-} mice.

FIGS. 2A-2E shows the results of phenotyping of mice with hepatocyte-specific deficiency of PKCβ. FIG. 2A shows a comparison of body weights at different ages in mice maintained on a chow diet. FIG. 2B shows plasma phospholipid, cholesterol, and triglyceride levels in the chow fed animals. FIG. 2C shows hepatic triglyceride levels in the chow fed animals. FIG. 2D shows hepatic cholesterol levels in the chow fed animals. FIG. 2E shows blood glucose levels in the chow fed animals.

FIGS. 3A-3D shows that hepatocyte-specific PKCβ deficiency does not affect diet-induced weight gain. FIG. 3A shows body weights of PKCβ^{fl/fl} and PKCβ^{Hep-/-} mice maintained on a high-fat and high-cholesterol diet (HFHC; 45 kcal % fat supplemented with 1% cholesterol) beginning 8 weeks of age. FIG. 3B representative images of the PKCβ^{fl/fl} and PKCβ^{Hep-/-} mice. FIG. 3C shows a comparison of weights of different tissues from PKCβ^{fl/fl} and PKCβ^{Hep-/-} mice fed a HFD for 12 weeks. FIG. 3D shows a comparison of food intake (g/mouse/weekly) in PKCβ^{fl/fl} and PKCβ^{Hep-/-} mice maintained on a high fat diet.

FIGS. 4A-4F shows that hepatocyte-specific PKCβ deficiency protects mice from HFD-induced hepatic steatosis in PKCβ^{fl/fl} and PKCβ^{Hep-/-} mice fed a HFD for 12 weeks. FIG. 4A shows representative sections of liver, WAT, and skin stained with H&E. FIG. 4B shows representative sections of liver stained with Oil Red. FIG. 4C shows a comparison of liver triglyceride. FIG. 4D shows a comparison of liver cholesterol FIG. 4E shows a comparison of plasma triglyceride. FIG. 4F shows a comparison of plasma cholesterol.

FIGS. 5A-5D shows that hepatocyte PKCβ deficiency does not affect glucose homeostasis. FIG. 5A shows western blot analysis of pooled liver lysates following IP insulin injection. FIG. 5B shows western blots for phospho-mTOR, phospho-SGK1, mTOR, GbL and Raptor levels in the liver of HFHC-fed mice injected with insulin in PKCβ^{fl/fl} and PKCβ^{Hep-/-} mice. FIG. 5C shows a comparison of fasted blood glucose levels in HFHC-fed PKCβ^{fl/fl} and PKCβ^{Hep-/-} mice. FIG. 5D shows the representative results of insulin tolerance test (ITT) in HFHC-fed PKCβ^{fl/fl} (dashed) and PKCβ^{Hep-/-} (solid) mice.

FIGS. 6A-6B shows that hepatocyte-specific PKCβ deficiency improves mitochondrial function in the liver. FIG. 6A shows an electron flow assay (EF) oxygen consumption rate (OCR) in isolated mitochondria from liver of PKCβ^{fl/fl} and PKCβ^{Hep-/-} mice fed on a HFHC diet, in the absence or presence of indicated substrates, inhibitors, or modulator of electron transport chain. FIG. 6B shows an electron coupling assay (EC) oxygen consumption rate (OCR) in isolated mitochondria from liver of PKCβ^{fl/fl} and PKCβ^{Hep-/-} mice fed on a HFHC diet, in the absence or presence of indicated substrates, inhibitors, or modulator of electron transport chain.

FIGS. 7A-7D shows the effects of hepatocyte-specific PKCβ deficiency on hepatic fatty acid synthase expression, SREBP-1 c processing, SREBP-1 c transactivation potential, and plasma VLDL levels. FIG. 7A shows a comparison of hepatic fatty acid synthase and stearoyl coenzyme desaturase 1 expression between PKCβ^{fl/fl} and PKCβ^{Hep-/-} mice maintained on a high fat diet. FIG. 7B shows an immunoblot assay of pooled total cell extracts from liver of the mice described in FIG. 7A. FIG. 7C shows a luciferase assay demonstrating that PKCβ activates transcriptional activation potential of SREBP-1c, independent of ERK-1/2 and AKT. FIG. 7D shows triglyceride-lipoprotein VLDL distribution in pooled plasma from control and PKCβ^{Hep-/-} mice fed a HFD for 12 weeks.

FIGS. 8A-8B shows a lipidomic analysis of livers from PKCβ^{fl/fl} and PKCβ^{Hep-/-} mice maintained on a HFHC diet. FIG. 8A shows a comparison of liver mass contents of TG. FIG. 8B shows a comparison of individual molecular species of TG.

FIGS. 9A-9C shows a comparative analysis of cardiolipin in the liver of PKCβ^{fl/fl} and PKCβ^{Hep-/-} mice maintenance on a high fat diet. FIG. 9A shows a comparison of total cardiolipin levels. FIG. 9B shows a comparison of total lysocardiolipin (LCL). FIG. 9C shows a comparison of cardiolipin molecular species.

FIGS. 10A-10C shows structures of PKCβ inhibitors. FIG. 10A shows the structure of Ruboxistaurin hydrochloride (LY333531). FIG. 10B shows the structure of INST3399 (HW-6-535-4). FIG. 10C shows the structure of INST5660 (HW-2-116-4).

FIG. 11 shows a representative western blot analysis for phosphorylated Histone H3 (P^{Ser10}-Histone H3) by recombinant and purified PKCb in the absence or presence of indicated inhibitors.

FIGS. 12A-12I demonstrates the efficacy of PKCβ inhibitors on diet-induced obesity. FIG. 12A shows body weight gain in mice maintained on a high-fat diet (HFD; 60% kcal from fat) in the absence or presence of LY333531 or INST3399, administered via intraperitoneal route. FIG. 12B shows body weight change in mice maintained on a HFHC diet in the absence or presence of INST3399. As expected from above studies, the data in FIG. 12C shows the effect of intraperitoneal INST3399 and LY333531 on weight gain in mice maintained on a HFHC diet. FIG. 12D shows the effect of oral INST3399 or LY333531 on weight gain in mice maintained on a HFHC diet. FIG. 12E compares food intake in mice fed HFD for and the indicated PKCb inhibitor was administered by i.p. FIG. 12F compares food intake in mice fed HFD and the indicated PKCb inhibitor was administered orally by gavage. FIG. 12G shows the ability of INST5660 in reducing diet-induced obesity in mice. FIG. 12H shows that INST3399 administration also reduces liver TG levels. FIG. 12I shows the ability of INST5660 in reducing diet-induced liver triglyceride level in mice.

FIG. 13 shows a timeline for analyzing N-nitrosodiethylamine (DEN)-induced Hepatocellular carcinoma (HCC) in mice fed HFHC diet.

FIG. 14A-14B shows anatomical analysis of liver from animals treated with DEN followed by HFHC diet. FIG. 14A shows nodules and hepatocellular carcinoma in DEN-treated PKCβ^{fl/fl} mice fed HFHC diet, whereas FIG. 14B shows lack of nodules and hepatocellular carcinoma in PKCβ^{Hep-/-} mice.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.”

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure. For example, a therapeutic effective dose of about 0.5 mg/kg body weight to about 4 mg/kg body weight includes 0.45 mg/kg to 4.4 mg/kg.

As used herein, the term “subject” shall refer to a mammal, preferably a human.

As used herein, the term “therapeutically effective amount” refers to the concentration of the inhibitor or other compound that is sufficient to elicit the desired therapeutic effect. It is generally understood that the therapeutically effective amount of the inhibitor will vary according to the weight, sex, age and medical history of the subject. Other factors which influence the therapeutically effective amount may include, but are not limited to, the severity of the patient's condition, the disease or disorder being treated, the stability of the inhibitor and, if desired, another inhibitor or another type of therapeutic agent being administered with the inhibitor(s) of the invention. A larger total dose may be delivered by multiple administrations of the inibitor. Methods to determine efficacy and dosage are known to those skilled in the art.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the pharmaceutical compositions is contemplated.

As used herein, the terms “parenteral administration” or “administering parenterally” refer to routes of administration that are other than via the gastrointestinal tract.

As used herein, the terms “protein kinase Cβ inhibitor”, “inhibitor” and “derivative or analog of bisindolylmaleimide” are interchangeable and refer to the therapeutically effective compounds or agents described herein.

In one embodiment of the present invention there is provided a protein kinase Cβ inhibitor with a chemical structure

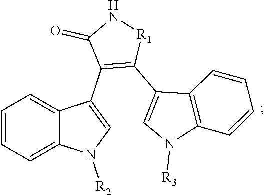

or a pharmaceutically acceptable salt thereof; wherein, R₁ is C═O or N; and R₂ and R₃ independently are H, CH₃, or (CH₂)₄C≡N.

In this embodiment the chemical structure is a derivative or analog of bisindolylmaleimide. In one aspect of this embodiment the chemical structure may be

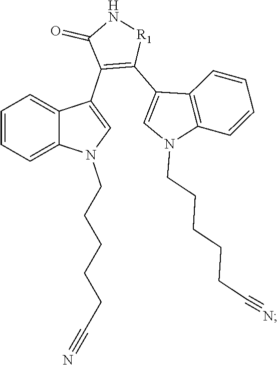

or a pharmaceutically acceptable salt thereof. In another aspect the chemical structure may be

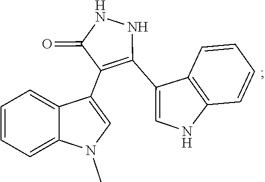 [SMILES: Cn1cc(-c2c(-c3c[nH]c4ccccc34)[nH][nH]c2=O)c2ccccc21]

or a pharmaceutically acceptable salt thereof.

In another embodiment of the present invention there is provided a pharmaceutical composition comprising at least one protein kinase Cβ inhibitor as described supra and a pharmaceutically acceptable carrier. Further to this embodiment the at least one protein kinase Cβ comprises a bisindolylmaleimide analog wherein R₁ and R₂ together with the indolyl nitrogens form a (dimethylamino)methyl-oxa-triazahexacyclic ring or a pharmaceutically acceptable salt thereof. In this further embodiment the bisindolylmaleimide analog may have the chemical structure

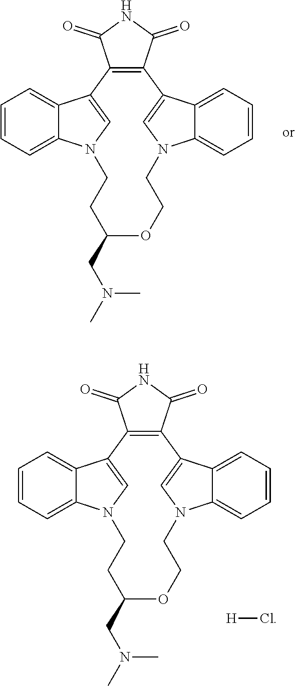 [SMILES: CN(C)C[C@H]1CCn2cc(c3ccccc32)C2=C(C(=O)NC2=O)c2cn(c3ccccc23)CCO1 ; CN(C)C[C@H]1CCn2cc(c3ccccc32)C2=C(C(=O)NC2=O)c2cn(c3ccccc23)CCO1 ; Cl]

In yet another embodiment of the present invention there is provided a method for treating obesity or an obesity-related disease in a subject in need of such treatment, comprising administering to the subject one or more times a therapeutically effective dose of the pharmaceutical composition as described supra. In this embodiment the obesity-related disease may be hepatic steatosis, nonalcoholic steatohepatitis or hepatocellular carcinoma. Also in this embodiment the therapeutically effective dose may be about 0.5 mg/kg body weight to about 4 mg/kg body weight. In addition the therapeutically effective dose may be administered daily or every other day.

In yet another embodiment of the present invention there is provided a method for treating a subject suffering from a metabolic disease, comprising the step of administering at least once to the subject, in a pharmaceutically acceptable carrier, a therapeutically effective dose of at least one protein kinase Cβ inhibitor with a chemical structure

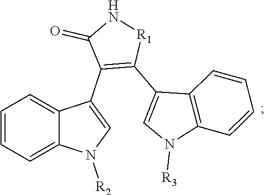

or a pharmaceutically acceptable salt thereof; wherein, R₁ is C═O or N; R₂ and R₃ independently are H, CH₃, or (CH₂)₄C≡N or R₁ and R₂ together with the indolyl nitrogens form a (dimethylamino)methyl-oxa-triazahexacyclic ring.

In this embodiment R₂ may be CH₃ and R₃ is H, R₁ and R₂ are each (CH₂)₄C≡N or R₁ and R₂ together form the a (dimethylamino)methyl-oxa-triazahexacyclic ring with the indolyl nitrogens. In one aspect of this embodiment the chemical structure may be

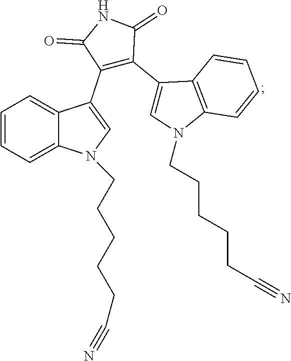 [SMILES: N#CCCCCCn1cc(C2=C(c3cn(CCCCCC#N)c4ccccc34)C(=O)NC2=O)c2ccccc21]

or a pharmaceutically acceptable salt thereof. In another aspect the chemical structure may be

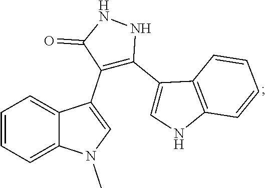 [SMILES: Cn1cc(-c2c(-c3c[nH]c4ccccc34)[nH][nH]c2=O)c2ccccc21]

or a pharmaceutically acceptable salt thereof. In yet another aspect the chemical structure may be

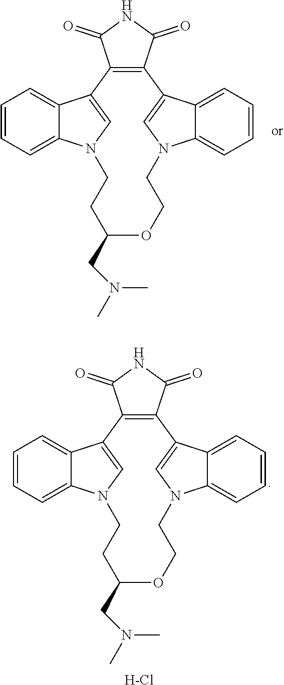

In this embodiment and all aspects thereof the metabolic disease may be obesity or an obesity-related disease or a combination thereof. Particularly, the obesity-related disease may be hepatic steatosis, nonalcoholic steatohepatitis or hepatocellular carcinoma. Also in this embodiment and all aspects thereof the therapeutically effective dose and rate of administration thereof is as described supra.

In yet another embodiment of the present invention there is provided a method for treating at least one of obesity or other obesity-related disease in a subject in need thereof, comprising the step of administering, parenterally, at least once to the subject, in a pharmaceutically acceptable carrier, a therapeutically effective dose of at least one protein kinase Cβ inhibitor with a chemical structure

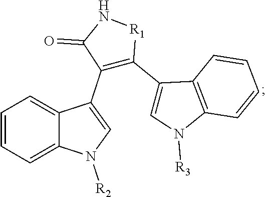

or a pharmaceutically acceptable salt thereof; wherein, R₁ is C═O or N; R₂ and R₃ independently are H, CH₃, or (CH₂)₄C≡N or R₁ and R₂ together with the indolyl nitrogens form a (dimethylamino)methyl-oxa-triazahexacyclic ring.

In this embodiment method of claim 21, wherein the protein kinase Cβ inhibitor or a pharmaceutically acceptable salt thereof has the chemical structure

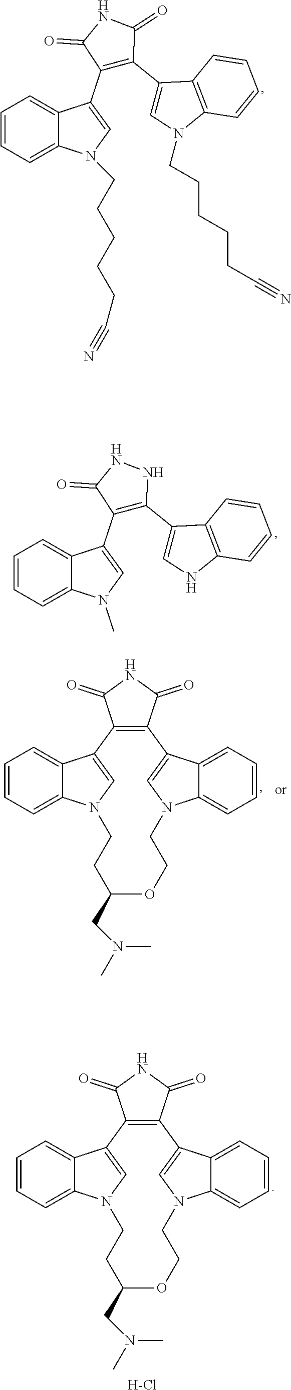

In one aspect of this embodiment the step of administering may comprise administering the therapeutically effective dose of the at least one protein kinase Cβ inhibitor intraperitoneally. Particularly, in this aspect the therapeutically effective dose may be about 0.5 mg/kg body weight to about 4 mg/kg body weight. In another aspect the step of administering may comprise administering the therapeutically effective dose of the at least one protein kinase Cβ inhibitor daily or every other day. In this embodiment and both aspects the obesity-related disease may be hepatic steatosis, nonalcoholic steatohepatitis or hepatocellular carcinoma.

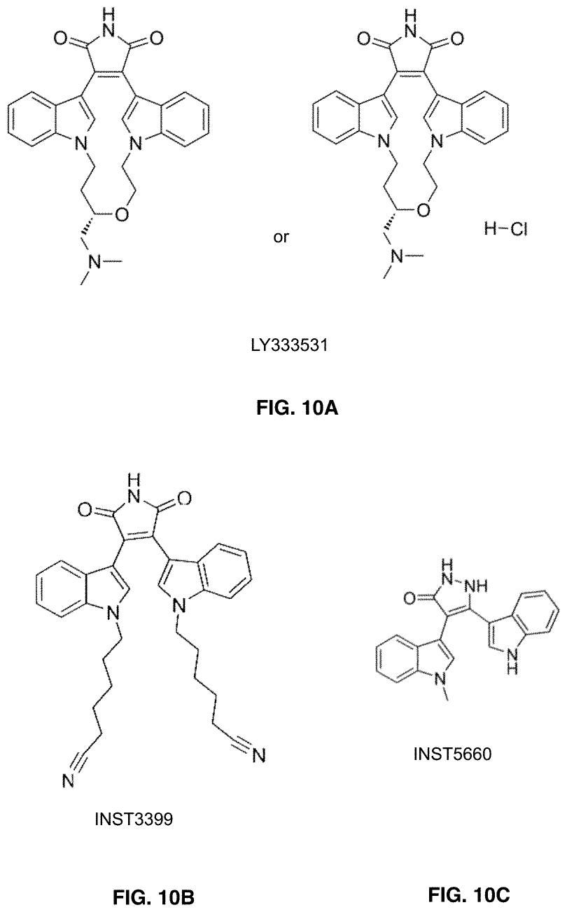

Provided herein are protein kinase Cβ inhibitors, for example derivatives or analogs of bisindolylmaleimide. Non-limiting examples of these inhibitors are structurally shown and described herein (FIGS. 10A-10C). The protein kinase Cβ inhibitors may be formulated in a salt form, for example, as an inorganic salt form, preferably, but not limited to, as a hydrochloride salt. Other acceptable salts are well-known in the art. The protein kinase Cβ inhibitors may be formulated in a pharmaceutically acceptable carrier with or without an inert diluent.

Also provided are methods for treating a metabolic disease in a subject suffering from the metabolic disease or otherwise in need of such treatment. The metabolic disease may be obesity or an obesity-related disease. For example, the obesity-related disease may be hepatic steatosis, commonly known as fatty liver disease, nonalcoholic steatohepatitis or hepatocellular carcinoma.

The treatment may comprise administering one or more times to the subject at least one of the protein kinase Cβ inhibitors, including bisindolylmaleimide, or a pharmaceutically acceptable salt thereof. The inhibitors may be formulated as described, for example, as a salt or with a pharmaceutically effective carrier as a pharmaceutical composition. Except insofar as any conventional carrier or other media, agent, or diluent is detrimental to the subject or to the therapeutic effectiveness of the formulation contained therein, its use in an administrable formulation for use in practicing the methods of the present invention is appropriate. The protein kinase Cβ inhibitors or formulations or pharmaceutical compositions thereof are suitable for parenteral administration, for example, but not limited to, intraperitoneal administration.

The protein kinase Cβ inhibitors or formulations or pharmaceutical compositions thereof may be administered at a dose and on a schedule one of ordinary skill in the art is well able to determine. For example, but not limited to, administration preferably may be daily or every other day. A representative dose or dosage may be about 0.5 mg/kg body weight to about 4 mg/kg body weight. The concentration of the protein kinase Cβ inhibitor in the pharmaceutical composition may from about 1 μM to about 3 μM.

The following examples are given for the purpose of illustrating various embodiments of the invention and are not meant to limit the present invention in any fashion.

### EXAMPLE 1

### Generation of Floxed PKCβ (PKCβ^{fl/fl}) and Hepatocyte-specific PKCβ-Deficient (PKCβHep^{-/-}) Mouse Models

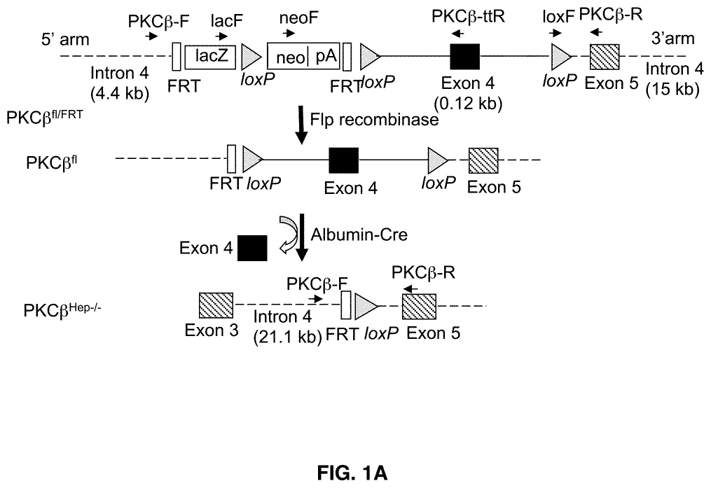

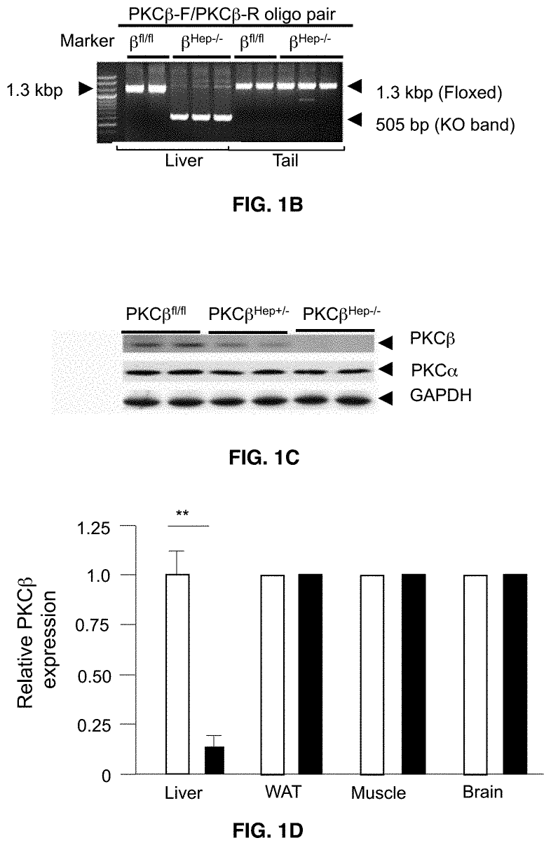

FIG. 1A shows the targeting strategy used to generate PKCβ^{fl/fl} and PKCβ^{Hep-/-} mice. Maps of the PKCβ genomic locus showing the conditional allele (upper panel) and the knockout allele (lower panel). The arrowheads indicate the loxP sites and the boxes represent the respective exons. PKCβ^{fl/fl} mice were generated through homologous recombination. Exon4 of the PKCβ gene was flanked by two loxP sites. PKCβ conditional knockout mice in C57BI/6 background were generated at the Ohio State University Comprehensive Cancer Center Genetically Engineered Mouse Modeling Core Facility by standard embryonic stem (ES) cell technology. The ES JM8.N4 clone EPD0744_4_5H11 was acquired from the International Mouse Phenotyping Consortium (IMPC). These cells carry the knock-out first tm1a Prkcb^{tm1a(EUCOMM)Wtsi} allele (IMPC Project #28059; www.mousephenotype.org). Chimeric males were bred to C57BL/6 Albino females and germline transmission was verified by PCR to detect the mutant together with the wild-type allele in the F1 heterozygous mice. Prior to utilization of the strain for experiments, mice were crossed to a FLPe ubiquitous strain (ACTB:FLPe B6J, Jackson Laboratory strain #005703) to eliminate the lacZ/neo cassette and obtain the clean tmlc allele according to the breeding schemes recommended by the IMPC.

To inactivate PKCβ in hepatocytes, PKCβ^{fl/fl} mice were crossed with Albumin-Cre transgenic mice in the C57BL6J genetic background. The littermates were screened by genotyping, and mice with two copies of loxP sites and Cre recombinase were characterized as PKCβ^{Hep-/-}. These mice were backcrossed 8 generations to C57BL/6J, and genetic background was verified using SNP genome scanning (143 SNP Panel; The Jackson Laboratory). FIG. 1B shows the validation of effective DNA recombination by PCR analysis of genomic DNA. FIG. 1C shows immunoblot analysis for PKCβ expression. FIG. 1D shows relative PKCβ expression in liver, white adipose tissue (WAT), muscle and brain from PKCβ^{fl/fl} and PKCβ^{Hep-/-} mice. Male mice were used in all experiment. PKCβ^{fl/fl} and PKCβ^{Hep-/-} mice were bred and maintained on a 12-hr-light and 12-hr-dark cycle with lights on from 7.00 am to 7.00 pm. All mice were given standard food pellets (normal chow diet) and water ad libitum. Cohorts of age-matched male mice were used for the study. Body weight and food intake were measured weekly. For high-fat diet (HFD) feeding experiments, mice were maintained on chow (15% kcal from fat; 7912 rodent chow; Harland Tekland, Wis.). For obesity experiments, mice were fed either HFD (60% kcal from fat; D12492; Research Diets, New Brunswick, NJ) or HFHC diet (45% kcal from fat supplemented with 1% cholesterol; #D04102102, Research Diets, New Brunswick, N.J.) beginning at the age of 6-8 wk. The institutional Animal Care and Use Committee at the Ohio State University Care Facility approved all studies using animal protocol.

### Mitochondrial Isolation and Respiration Function Analysis

Mitochondria were isolated as described (20, 22). Four micrograms of isolated mitochondria from liver, gastrocnemius/plantaris muscle, BAT, and iWAT were resuspended in respiratory assay buffer composed of 70 mM Sucrose, 220 mM mannitol, 10 mM K₂HPO₄, 5 mM MgCl₂, 2 mM HEPES, and 1 mM EGTA, pH 7.4). Electron coupling and electron flow assays were performed using the Seahorse Bioanalyzer. Briefly, mitochondria were incubated with the indicated substrates and oxygen consumption rates were determined. Mitochondria basal respiration in electron coupling assays was determined in a coupling state with 10 mM Succinate initial substrate with 2 μM Rotenone. State 3 respiration was initiated with the injection of ADP, State 4 respiration was initiated with the injection of Oligomycin, and maximal uncoupler-stimulated respiration was initiated with the injection of FCCP (Trifluoromethoxy carbonylcyanide phenylhydrazine). Mitochondrial basal respiration in electron flow assays was determined in an uncoupled state with initial substrates 10 mM pyruvate and 2mM malate in the presence of FCCP. Sequential electron flow throughout the electron transport chain was determined by first injecting Rotenone, followed by Succinate, Antimycin A, and Ascorbate and TMPD (N,N,N′,N′-Tetramethyl-p-phenylenediamine).

### Measurement of SREBP-1c Transactivation

(Gal4)₅-luciferase reporter plasmid (0.6 μg) was co-transfected with a plasmid encoding either Gal4-DNA binding region (Gal4-DBD) or activation domain of SREBP-1 c linked to Gal4-DNA binding domain (Gal4-DBD-SREBP-1AD) (0.3 μg) (23) and pCMV-8-galactosidase (0.1 μg), along with constitutively active PKCβ cDNA (0.1 μg), in human hepatoma HepG2 cells in the absence or presence of LY333,531 (5 μM), PD98059 (20 μM) or GSK690693 (1 μM). Fold induction represens luciferase activity on PKCβ transfection relative to basal expression level in the absence of PKCβ expression vector (taken as 1). Luciferase activity was normalized to β-galactosidase activity.

### Shotgun Lipidomics Analysis

Cell pellets were homogenized in 0.5 mL of 10× diluted PBS in 2.0-mL cryogenic vials (Corning Life Sciences) by using a digital sonifier (Branson 450). For shotgun lipidomics, lipid extracts were diluted to a final concentration of ˜500 fmol/μL, and the mass spectrometric analysis was performed on a QqQ mass spectrometer (Thermo TSQ Quantiva) equipped with an automated nanospray device (TriVersa NanoMate; Advion Bioscience Ltd.) as previously described (24). Identification and quantification of all of the reported lipid molecular species were performed using an in-house automated software program following the principles for quantification by MS as previously described (25). Fatty acyl chains of lipids were identified and quantified by neutral loss scans or precursor ion scans of corresponding acyl chains and calculated using the same in-house software program. Data were normalized to per milligram of protein. Lipid Internal Standards:1,2-Dimyristoleoyl-sn-glycero-3-phosphocholine (di14:1 PC) (All of the lipid internal standards are purchased from Avanti Polar Lipids, Inc., Alabaster, Ala.).

### Histology

Liver, WAT and BAT from ad libitum fed mice were isolated and fixed in 4% paraformaldehyde and processed for H& E staining. For Oil Red O staining, liver tissues were fixed in 4% paraformaldehyde overnight and incubated in 12% sucrose for 12 h and then in 18% sucrose overnight before being cryo-embedded and sectioned.

### Plasma and Tissue Chemistry

Blood was collected using a 1-mL syringe coated in 0.5 M K2EDTA, and serum was collected by centrifugation at 1000 g for 20 min. Insulin levels were measured by ELISA. Serum and liver TG, cholesterol, and lipoprotein distribution were measured by the Mouse Metabolic Phenotyping Core Facility at University of Cincinnati College of Medicine.

### Immunoblot Analysis

Proteins were extracted from liver tissue of mice (18, 20). Livers were homogenized in RIPA buffer, 10 mM NaF, 1 mM Na₃VO₄, 1 mM PMSF, and protease inhibitor tablet (Roche Diagnostic). Protein concentration was determined using a BCA protein assay kit (Thermo Scientific), and lysates were analyzed by SDS-polyacrylamide gel electrophoresis and Western blot analysis on a PVDF membrane. Antibody to PKCβ (F-7) was purchased from Santa Cruz Biotechnology, whereas antibodies to AKT (#4685), P-AKT^{Thr308} (#13038) P-AKT^{ser473} (#4060), Insulin receptor beta (#3025), P-Insulin receptor/IGF1R beta (#3021), P-IRS-1^{Ser307} (#2381), P-IRS-1^{Ser612} (#3203), P-IRS1^{Ser318} (#5610), IRS-2 (#4502), P-mTOR^{Ser2448} (#5536), p-mTOR^{Ser2481} (#2974), mTOR (#2983), rictor (#2114), and GβL (#3274) were purchased from Cell Signaling Technology, Danvers, Mass. Phospho-SGK1^{Ser422} (#55281) and SGK1 (#43606) were purchased from Abcam, Cambridge, Mass. Goat anti-mouse and goat anti-rabbit HRP-conjugated secondary antibodies (Bio-Rad) were used.

### In Vivo Insulin Signaling

Following an overnight fast, mice were anesthetized with 2,2,2-tribromoethanol in PBS and injected with 5 U of regular human insulin (Novolin, Novo Nordisk) via the inferior vena cava. Five minutes after the insulin bolus, tissues were removed and frozen in liquid nitrogen. Immunoblot analysis of insulin signaling molecules were performed using liver homogenates prepared in a tissue homogenization buffer that contained 25 mM Tris-HCl (pH 7.4), 10 mM Na₂VO₄, 100 mM NaF, 50 mM Na₃P₂O₇, 10 mM EGTA, 10 mM EDTA, 2 mM phenylmethylsulphonyl fluoride, and 1% Nonidet-P40 supplemented with protease inhibitor cocktail (Sigma-Aldrich). All protein expression data were quantified by densitometry using NIH Image.

### Insulin Tolerance Test

The Insulin tolerance test was performed as described (17, 18).

### Statistical Analysis

All values are given as mean standard error. Differences between two groups were assessed using unpaired two-tailed Student's t-tests. P<0.05 was regarded as significant. Statistical analysis was performed in Excel (Microsoft).

### EXAMPLE 2

### Hepatocyte-specific PKCβ Deficiency Protects Against Diet-induced Hepatic Steatosis

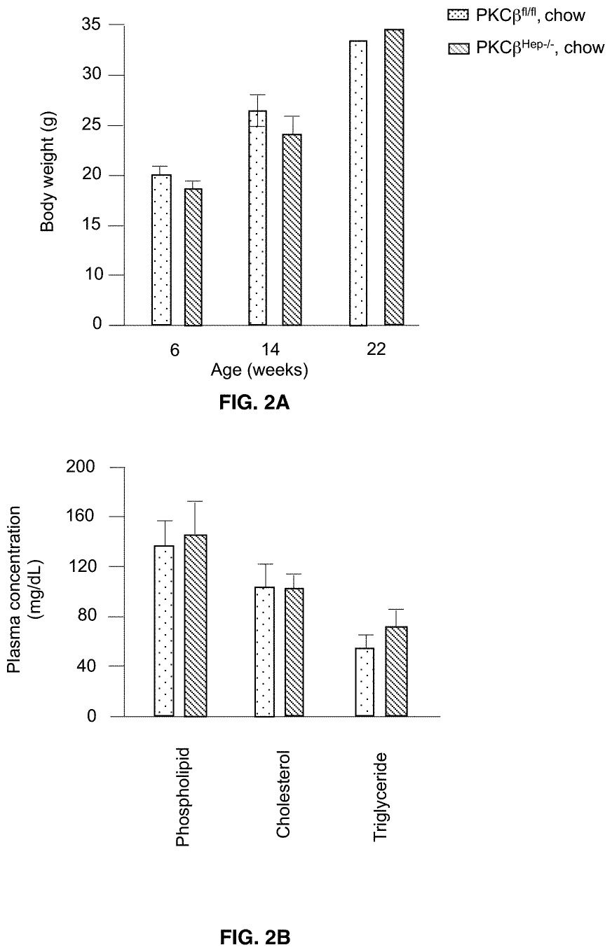

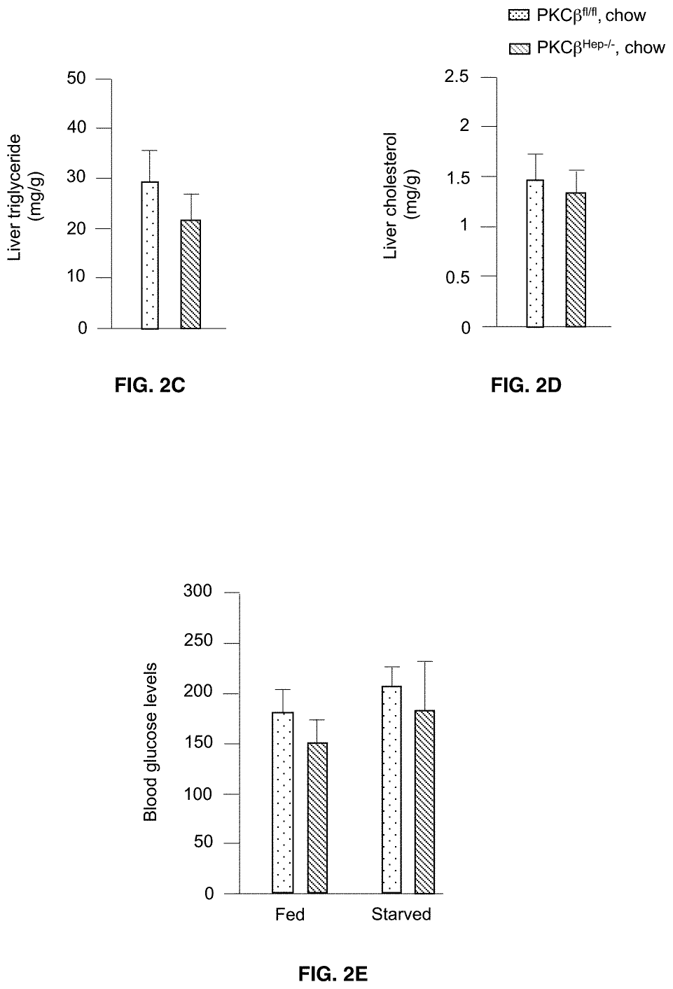

When maintained on normal chow ad libitum, PKCβ^{Hep-/-} mice exhibited similar body weight compared to control PKCβ^{fl/fl} mice (FIG. 2A). Gross metabolic comparisons between these mice revealed no significant differences in blood phospholipid, triglycerides and cholesterol (FIG. 2B). There was a slight but non-significant decrease in hepatic TG content, with no difference in hepatic cholesterol content of PKCβHep^{-/-} mice compared to control mice (FIGS. 2C and 2D). Comparison of blood glucose revealed no significant differences between genotypes (FIG. 2E). Thus, loss of the PKCβ in hepatocytes appears to exert no significant metabolic effect in mice maintained on normal chow.

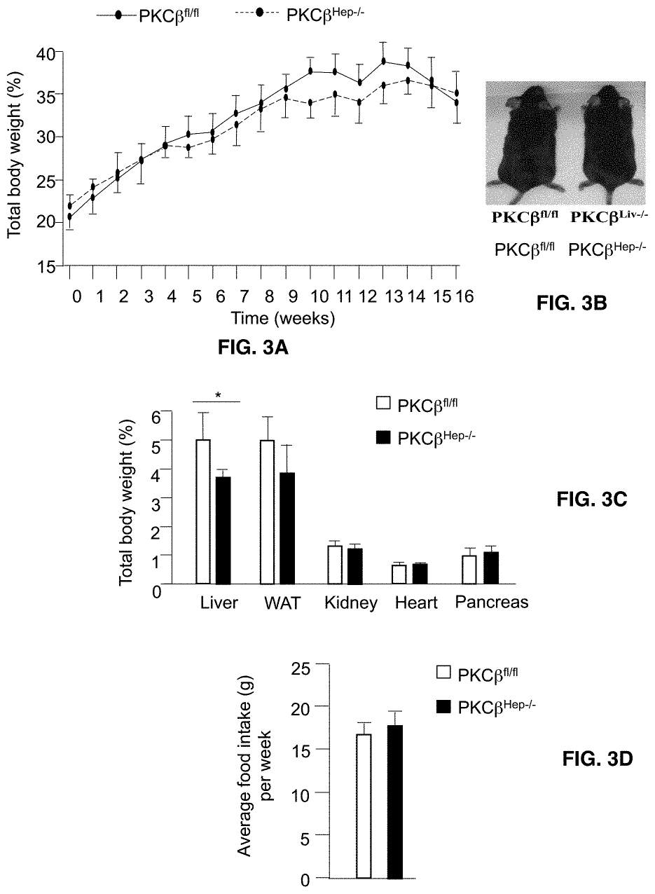

To understand how PKCβ deficiency might influence nutrient handling in mice upon chronic lipid overflow, PKCβ^{fl/fl} and PKCβ^{Hep-/-} mice were maintained on a high fat diet (HFD). After 12-16 weeks on HFD, PKCβ^{Hep-/-} and control mice showed similar weight gains (FIGS. 3A and 3B). Interestingly, liver was significantly smaller in these mice (FIG. 3C), with a non-significant decrease in epididymal white adipose tissue (eWAT), despite similar food intake (FIG. 3D). No differences were observed in kidney, heart and pancreas weights (FIG. 3C).

### HFHC-Induced Obesity Normally Leads to Lipids Accumulation in the Liver

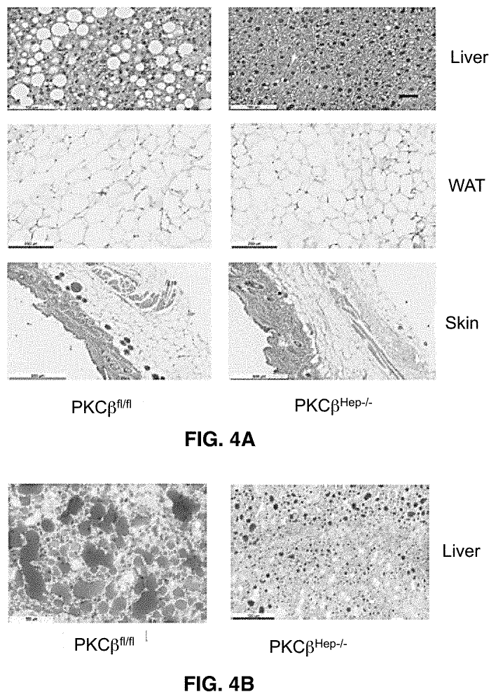

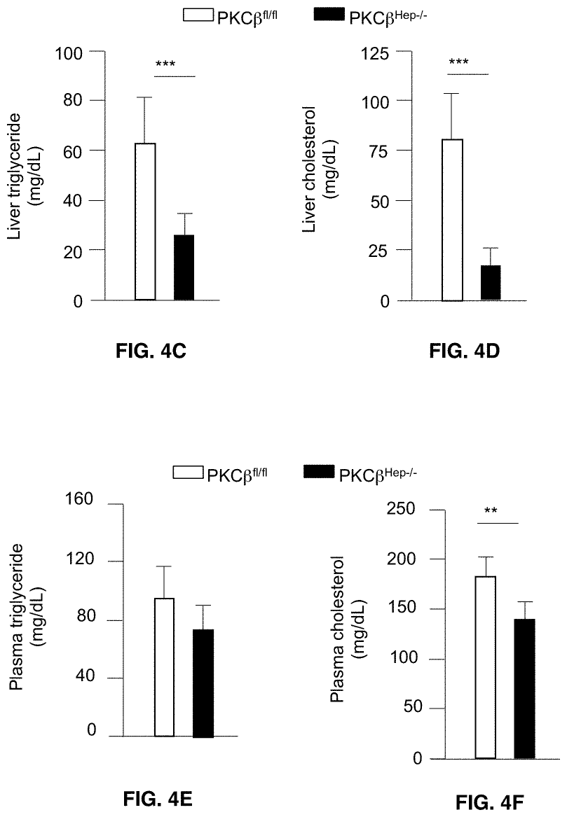

Histological examination of livers revealed reduced numbers and sizes of intracellular vacuoles—an indication of reduced fats—in PKCβ^{Hep-/-} mice compared to control PKCβ^{fl/fl} mice (FIG. 4A). Oil Red O staining of liver sections verified deposition of increasing quantities of lipids in control livers (FIG. 4B). Histological analysis of WAT revealed that adipocytes from WAT were slightly smaller than those from control mice (FIG. 4A). There was however no difference in the thickness of adipose tissue beneath the dermis between genotypes (FIG. 4A). As expected, based on histological analysis, biochemical measurements confirmed pronounced decrease in both TG and cholesterol levels in liver from HFHC-fed PKCβ^{Hep-/-} mice compared to control mice (FIGS. 4C and 4D). Decreased liver lipid content was found to be accompanied by significant reduction in plasma cholesterol, with slightly lower (but not significant) plasma triglycerides (FIGS. 4E and 4F). Together, these findings demonstrate that hepatocyte PKCβ deficiency protects mice from development of hepatic steatosis in response to caloric excess.

### Hepatocyte-Specific PKCβ Deficiency Does Not Protect Against Diet-Induced Insulin Resistance

There have been reports that PKCβ can phosphorylate insulin receptor and insulin receptor substrate-1 (IRS1), and protein kinase B (AKT) in various cell culture models (27-29). These in vitro experimental results have been conflicting, suggesting both negative and positive regulatory roles. To investigate the role of PKCβ in insulin signaling in the liver, fasted mice were injected with insulin or saline and analyzed for changes in phosphorylation of insulin signaling components.

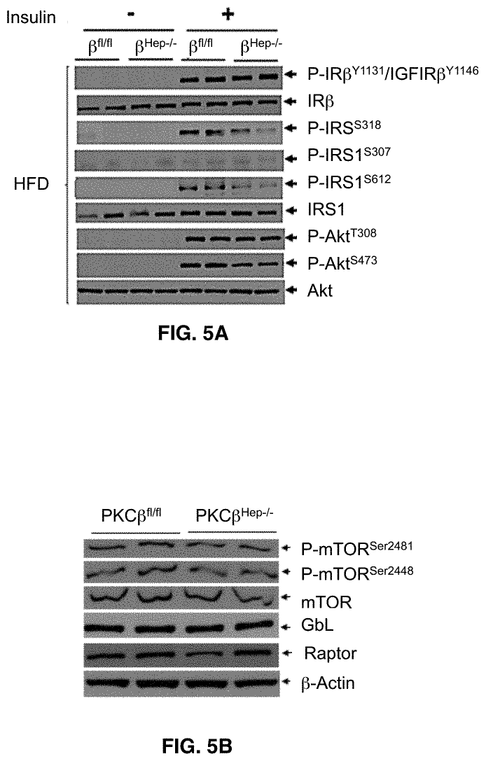

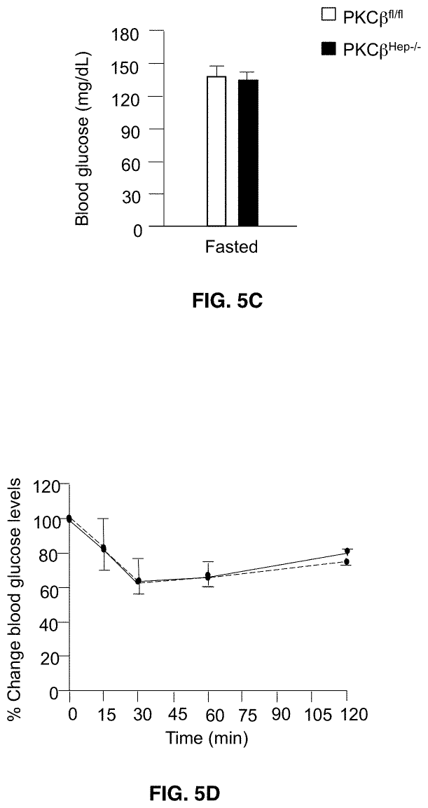

Insulin-induced tyrosyl phosphorylation of the insulin receptor was comparable in livers of control and PKCβ mice (FIG. 5A). No apparent effect of hepatocyte PKCβ deficiency on insulin-induced low-level phosphorylation of IRS1-Ser³⁰⁷ was observed, whereas insulin-stimulated IRS1-Ser³¹⁸ (−60±9% of control) and ΔSer⁶¹² (−50±14% of control) phosphorylation, normalized to expression of IRS1, were lower in PKCβHep^{-/-} livers compared to controls. Insulin-stimulated phosphorylation of AKT-Thr³⁰⁸ was similar, whereas mildly reduced phosphorylation of AKT-Ser⁴⁷³ (−25±7% of control) was observed in PKCβHep^{-/-} livers.

There are several mechanisms possible for PKCβ to regulate AKT-Ser⁴⁷³ phosphorylation. One possibility is that PKCβ acts as an AKT kinase or activates the mechanistic target of rapamycin (mTORC) to phosphorylate AKT on Serine⁴⁷³. mTORC1 and mTORC2 share mTOR protein which can be phosphorylated at several residues, including Thr²⁴⁴⁶, Ser²⁴⁴⁸ and Ser²⁴⁸¹. Phosphorylation of mTOR at Ser2481 distinguishes activated mTORC2 from activated mTORC1 (30).

To evaluate mTORC2 activity the phosphorylation status of mTORC2 and its substrate SGK1 were investigated in the liver of mice treated with insulin. Unlike AKT-Ser⁴⁷³ phosphorylation, no differences were observed in the phosphorylation levels of mTOR-Ser²⁴⁴⁶ and -Ser²⁴⁸¹ and phospho-SGK1-Ser⁴²² between genotypes (FIG. 5B). No significant difference in expression of mTORC2 components RICTOR and GβL were observed. These results support that hepatic PKCβ is not essential for AKT-Ser⁴⁷³ phosphorylation but may be required for its maximal activation in the liver in response to insulin.

Next, potential effects of hepatic PKCB deficiency on glucose homeostasis were investigated in vivo. Blood glucose levels were similar between genotypes (FIG. 5C), suggesting no major effect of hepatocyte PKCB deficiency on glucose homeostasis. Consistent with the above result, no differences were observed in insulin-tolerance tests (ITTs) between control and PKCβHep^{-/-} mice (FIG. 5D).

Diacylglycerol (DAG), an activator of PKCs, has been proposed to mediate lipid-induced hepatic insulin resistance (31). However, the importance of DAG in lipid-induced hepatic insulin resistance remains controversial. A recent report has connected membrane diacylglycerol levels through PKC to insulin resistance in Non-alcoholic fatty liver disease (NAFLD) (32), membrane DAG levels in livers of above mice were compared. No significant changes in membrane DAG levels (87±24 vs 82±19 pmoles/mg protein, n=4, p>0.05) were observed between genotypes. In short, these findings indicate that disruption of hepatocyte PKCβ has no major effect on insulin signaling and glucose homeostasis.

### Hepatocyte-Specific PKCβ Attenuates SREBP-1c Transactivation and Improves Mitochondrial Function

As a central regulator of lipid homeostasis, liver is responsible for orchestrating the synthesis of new fatty acids, their export and subsequent redistribution to other tissues, as well as their utilization as energy substrates. Altered lipid homeostasis in the liver is the pathophysiological hallmark of hepatic steatosis. The disruption of one or more of these pathways may precipitate the retention of fat within the liver and the subsequent development of hepatic steatosis.

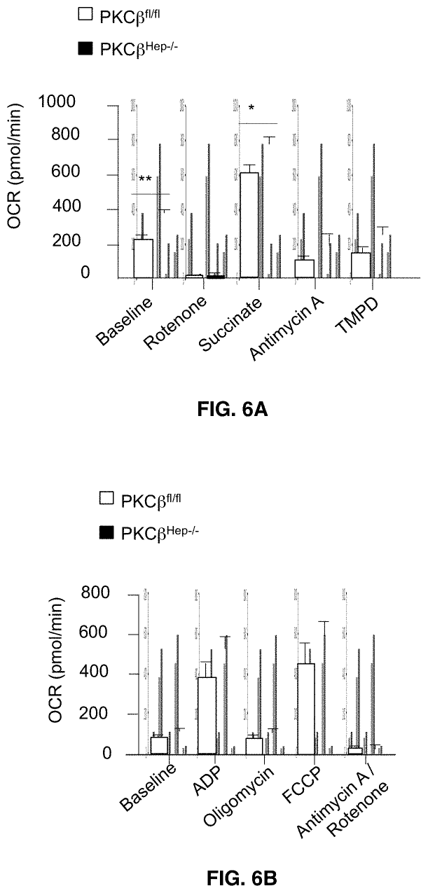

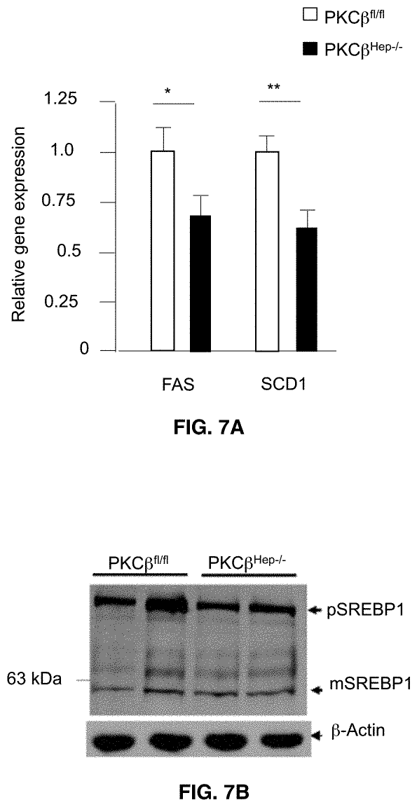

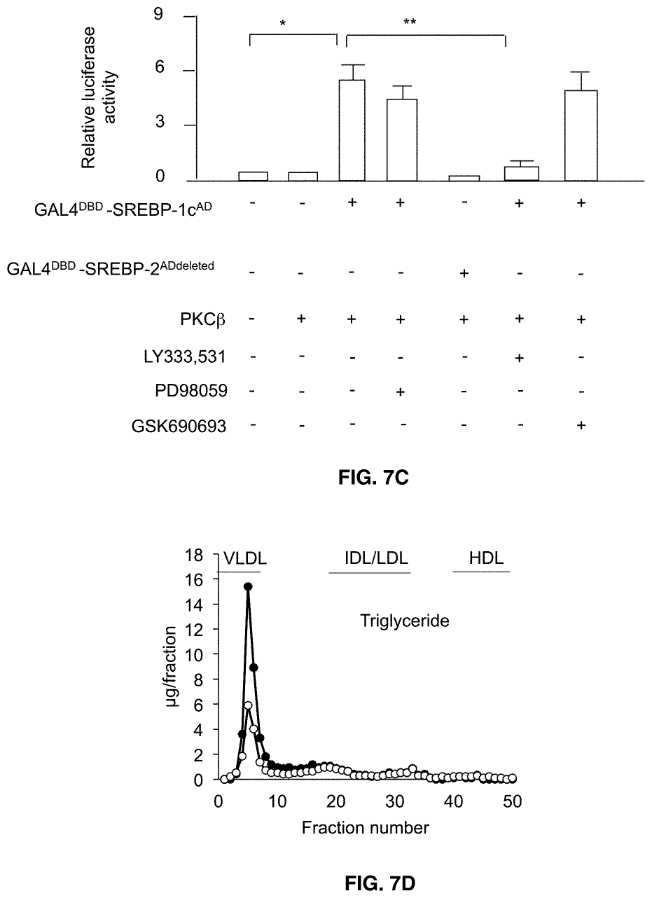

Healthy mitochondria are crucial for the adequate control of lipid metabolism in liver. To gain insight into the molecular impact of hepatocyte-specific PKCβ deficiency on mitochondrial metabolism, energetics in mitochondria isolated from livers of control and PKCβ^{Hep-/-} mice fed HFD were compared using a Seahorse XF analyzer. Under uncoupling condition, baseline oxygen consumption rates (OCRs) were significantly increased in liver mitochondria from PKCβ^{Hep-/-} mice and also in the presence of succinate (FIG. 6A). Under coupling conditions, there was an increase in liver mitochondria OCR with both adenosine diphosphate (ADP) and trifluoromethoxy carbonylcyanide phenylhydrazone (FCCP) compared to control, although this was not significant FIG. 6B). Improvement in mitochondrial function was also accompanied by reduced levels of fatty acid synthase and stearoyl CoA desaturase transcripts in liver of PKCβ^{Hep-/31} mice compared to control (FIG. 7A). The transcription factor sterol regulatory element-binding protein-1c (SREBP-1c) plays a central role in de novo fatty acid synthesis gene expression.

To first assess whether PKCβ deficiency affected SREBP-1c processing, precursor and nuclear forms of endogenous SREBP-1 in the liver of control and PKCβ^{Hep-/-} mice were compared. A slight reduction in expression of precursor SREBP-1 was observed. The nuclear levels of SREBP-1 were however similar in PKCβ^{Hep-/-} liver compared to control livers (FIG. 7B).

To determine whether PKCβ deficiency affected the activation of hepatic SREBP-1c, a plasmid was used in which activation domain of SREBP-1c is fused to the Gal4-DBD and evaluated the activation of a Gal4-responsive reporter plasmid by overexpressed PKCβ in the absence or presence of indicated inhibitor. Interestingly, PKCβ increased activation of SREBP-1c plasmid, and this activation was blocked by a specific inhibitor of PKCβ LY333,531, but not by either MEK inhibitor PD98059 or AKT inhibitor GSK690,693. These results support that PKCβ activates SREBP-1c through its amino terminal (FIG. 7C).

Lastly, to determine the potential effect of hepatocyte PKCβ deficiency on very-low density lipoprotein (VLDL) levels, plasma levels were compared between genotypes. There was a significant reduction in plasma VLDL levels in PKC8^{Hep-/-} mice compared to control suggesting that an increase in its production and secretion does not contribute to reduced hepatic steatosis in these mice (FIG. 7D). Thus, loss of hepatocyte PKCβ increases mitochondrial respiratory chain and lower SREBP-1c transactivation, which may account for reduced liver fat content in PKCβ^{Hep-/-} mice compared to control mice.

Hepatocyte-specific PKCβ Deficiency Leads to Elevated Liver Cardiolipin and Reduced Acylcarnitine Levels Commonly Associated with Fatty Liver Disease

Recent studies have underscored the importance of membrane lipids in mitochondrial function and in the pathophysiology of hepatic steatosis (33). In order to identify lipids discriminating the pathophysiological status of the liver in response to PKCβ deficiency, shotgun lipidomics analysis was performed on liver from WT and PKCβ^{Hep-/-} mice to compare fatty acyls, TG, acylcarnitine, cardiolipin, lysocardiolipin, and various phospholipids (phosphatidic acid, phosphatidylcholine, lysophosphatidylcholine, phosphatidylethanolamine, lysophosphatidyl-ethanolamine, phosphatidylglycerol, phosphatidylinositol, and phosphatidylserine).

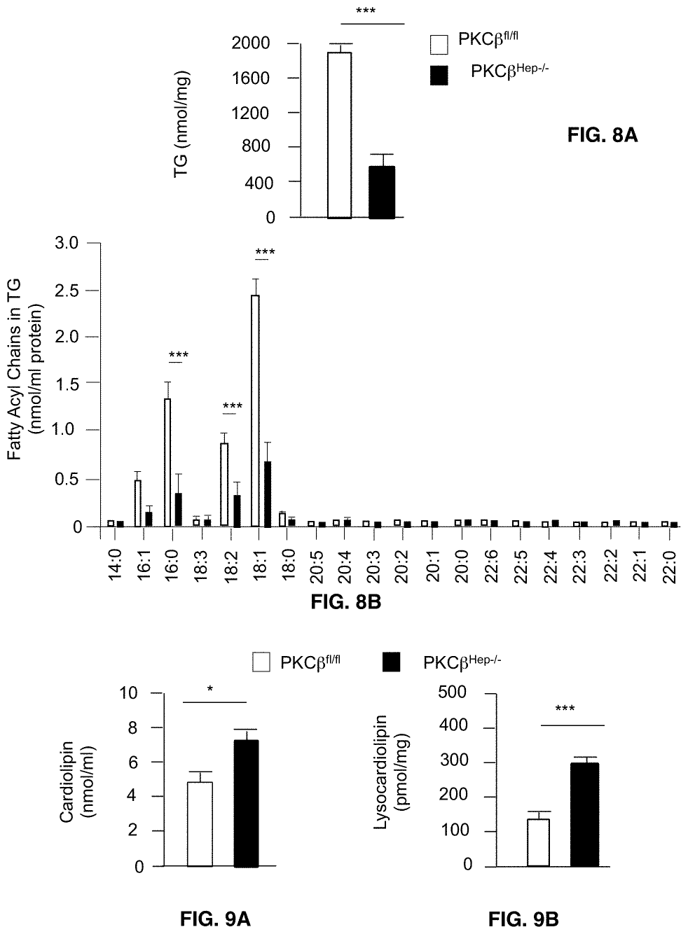

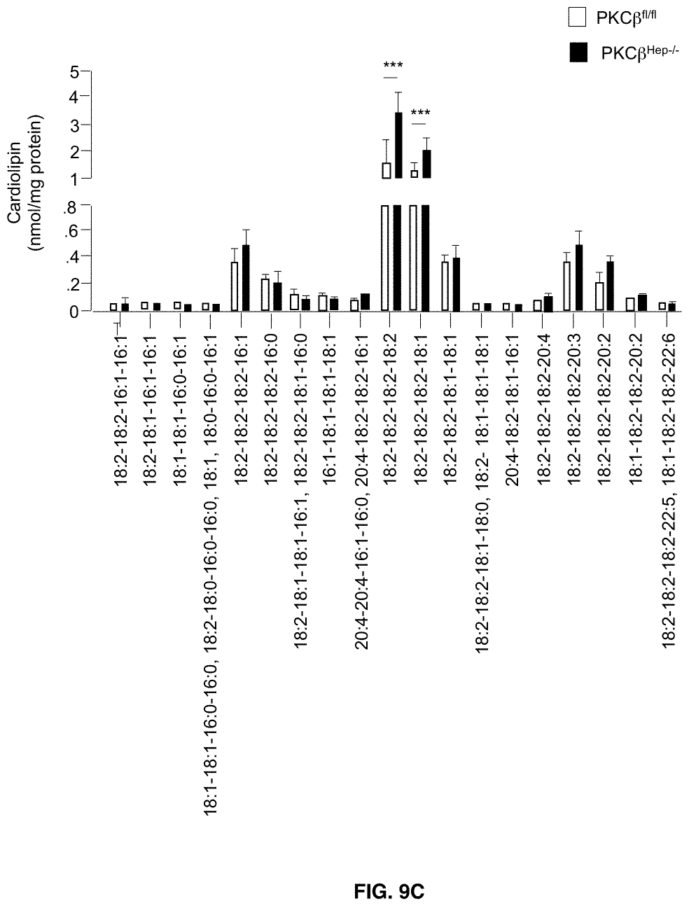

Consistent with the biochemical study, shotgun lipidomics identified a significant decrease in hepatic TG content in PKCβ^{Hep-/-} livers compared to control livers (FIG. 8A). There were however no specific changes in TG molecular species composition of TG between PKCβ^{Hep-/-} mice and WT counterparts. The decrease was significantly observed in TG molecular species (16:1), (16:0), (18:2), and (18:1) in PKCβ^{Hep-/-} livers compared to control livers (FIG. 8B). Similarly, livers from mice lacking PKCβ exhibited a reduction in levels of acylcarnitine (4.22+1.37 PKCβ^{fl/fl} versus 2.89±0.68 pmol/mg protein PKCβ^{Hep-/-}, n=4, p=0.18) which did not reach statistical significance. There were no other significant changes in liver sphingomyelin, phosphatidylethanolam ine, lysophosphatidylethanolam ine, phosphatidylcholine, lysophosphatidylcholine, phosphatidylglycerol, and phosphatidylinositol levels in the hepatic fatty acid fractions of the control and PKCβ^{Hep-/-} mice. However, hepatocyte PKCβ deficiency caused marked increases in both cardiolipin and lysocardiolipin (FIG. 9A). Importantly, cardiolipin comprises 10-20% of the mass of total mitochondrial phospholipid, and recent studies have correlated positively higher cardiolipin levels to improved mitochondrial membrane potential and respiration (34, 35). The biological function of this essential mitochondrial lipid is determined by the composition of its four fatty acyl chains as they control mitochondrial architecture and function (35).

Next, cardiolipin acyl composition in the same liver tissue samples was compared. Ablation of hepatocyte PKCβ predominantly elevated most abundant cardiolipin molecular species (18:2-18:2-18:2-18:2) and (18:2-18:2-18:2-18:1), whereas lysocardiolipin molecular species (18:2-18:2-18:2-18:1) specifically showed a significant increase (105.27±25.9 PKCβ^{fl/fl} versus 247±7.23 pmol/mg protein PKCβ^{Hep-/-}, n=4, ***p<0.001) and not lysocardiolipin (41.20±12.69 PKCβ^{fl/fl} versus 52.03±1.91 pmol/mg protein PKCβHep^{-/-}, n=4, p=0.212) (FIG.96). FIG. 9C shows multidimensional mass spectrometric array analyses (25) of cardiolipin molecular species from the livers of PKCβ^{Hep-/-} mice and PKCβ^{fl/fl} mice.

The above data indicate that hepatocyte PKCβ is a key focus of dietary lipid perception and is essential for efficient storage of dietary lipids in liver largely through coordinating energy utilization and lipogenesis during postprandial period. These results highlight the importance of hepatic PKCβ as a drug target for obesity-associated nonalcoholic hepatic steatosis.

### EXAMPLE 3

### Protein Kinase Cβ Inhibitors

FIG. 10A shows the structure of protein kinase Cβ inhibitor Ruboxistaurin hydrochloride (LY333531, Eli-Liily), which have been used in clinical trials for breast cancer, diabetic retinopathy and myopathy without any success. FIGS. 10B and 10C show respectively the structures of PKCβ inhibitors INST3399 (C₃₂H₃₁N₅O₂ Formula Weight 517.63) and INST5660 (C₂₀H₁₆N₄O Formula Weight 328.38), which comprise the instant invention.

INST3399 and INST5660 Inhibited PKCβ more Effectively than LY333531 (in the Absence of Lipid Activator)

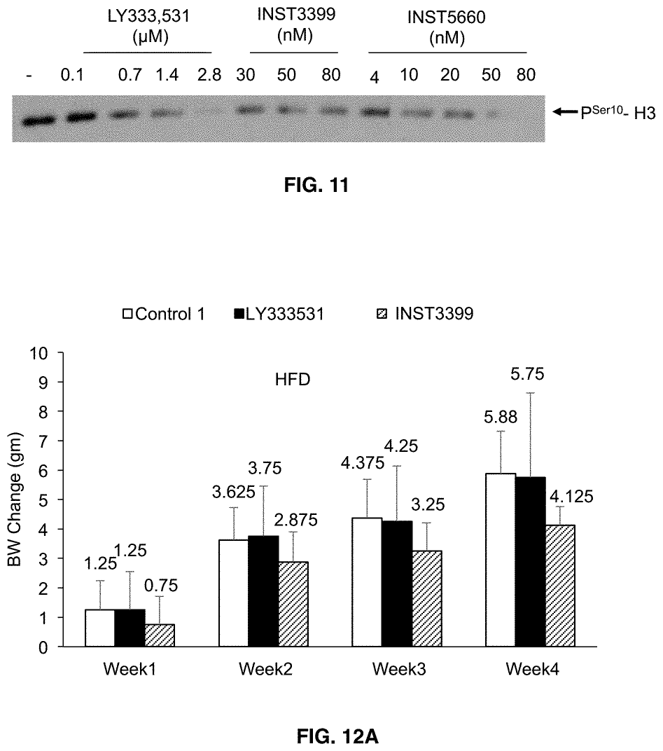

PKCb assay: This PKCb assay was used to compare PKCb inhibitory activity of LY333531, INST3399, and INST5660 (21). Recombinant PKCβ (11ng) was mixed with purified Histone H3 (20 ng) and incubated at 30° C. for 6 minutes and phosphorylated Histone H3 (P^{ser10}-Histone H3) assessed by immunoblotting using specific antibodies (FIG. 11). Lipid activator was not included our assay because we were interested in finding chemicals inhibiting PKCb in its basal state. IC₅₀ (50% inhibition) was calculated from the immunoblotting results and found to be as shown in Table 1. IC₅₀ of 700 nM is higher than previously reported value for LY333531 for PKCb because LY333531 inhibits PKCb most potently when this kinase is activated (36). It is clear from our study that both inhibitors INST3399 (IC₅₀ of 50 nM) and INST5660 (IC₅₀ of 10 nM) inhibit basal PKCb more potently than LY333531 in parallel experiments.

TABLE 1
IC₅₀ values for the tested inhibitors

| Inhibitor | IC₅₀ | |
|---|---|---|
| LY333531 | 700 ± 140 | nM |
| INST3399 | 50 ± 10 | nM |
| INST5660 | 10 ± 3 | nM |

### Efficacy of PKCβ Inhibitors on Diet-Induced Obesity and Hepatic Steatosis

To test and compare therapeutic efficacy of these inhibitors, the following experiment was set up in a C57BL6 mouse model (20 animals per study). C57BL6 mice (7 week old male mice) were fed either HFD or HFHC diet and were either untreated or treated on the same day with an indicated PKCb inhibitor. The PKCb inhibitor was administered to these mice either by i.p. or oral gavage.

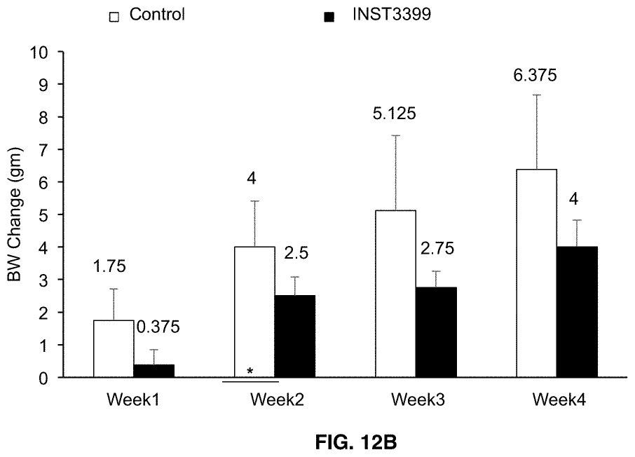

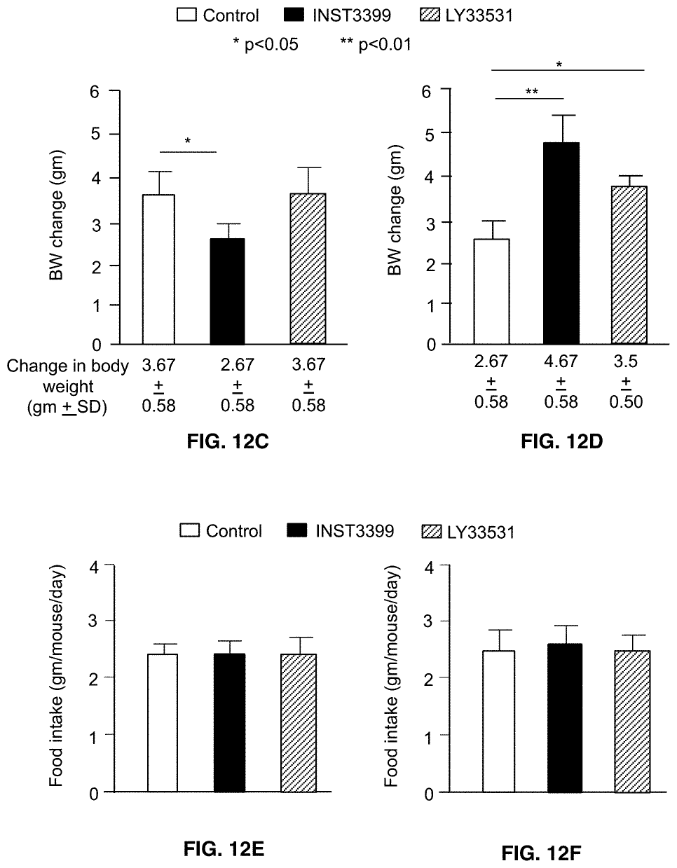

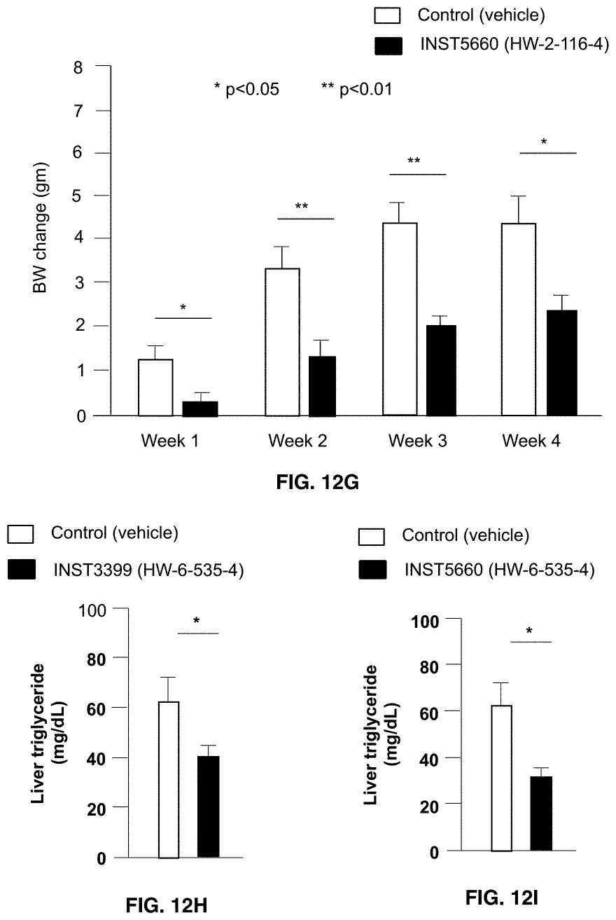

FIG. 12A shows body weight gain in mice maintained on a high fat diet (HFD, 60% fat) in the absence or presence of INST3399, administered via an intraperitoneal route (i.p). FIG. 12B shows body weight gain in mice maintained on a high fat high cholesterol (HFHC) (45% fat plus 1% cholesterol) in the absence or presence of INST3399, administered i.p. FIGS. 12C and 12D show the effects of administration of INST3399 and LY333531 on weight gain in mice maintained on a HFD for 1 week. The inhibitors were administered by i.p. (FIG. 12C) or gavage (FIG. 12D). A summary of the results for INST3399 or INST5660 treated animals fed with a HFD or a HFHC diet is shown in FIGS. 12E-12I. From FIGS. 12C and 12D, it is clear that intraperitoneal route of administration is central to a reduction in weight gain since oral administration of INST3399 or LY333531 in mice maintained on a high fat diet caused an increase in weight gain. As expected, INST3399 resulted in greater weight gain relative to LY333531 because INST3399 is a more potent PKCβ inhibitor than LY333531.

Hepatocyte-Specific PKCβ Deficiency Protects Mice from Diet-Induced Hepatic Steatosis, and Hepatocellular Carcinoma (HCC)

Diethylnitrosamine (DEN) treatment. Groups of 10-day-old control PKCβ ^{fl/fl} and PKCβ^{Hep-/-} mice mice were administered a single intraperitoneal injection of the genotoxic hepatocarcinogen, DEN (Sigma-Aldrich, Mo.) dissolved in PBS at a dose of 20 mg/kg body weight, or saline (control). Mice were fed HFHC diet (45% fat and 1% cholesterol) and sacrificed respectively at week 28 following DEN or saline injections. The livers were weighed, the diameter of tumors mm on the surface of the livers was enumerated and the sizes of tumors were measured. A portion of the liver tissue was fixed in AAF (100% alcohol 85 ml, acetic acid 5 ml, formalin 10 ml) for histological study, while the rest was frozen at −80° C. until use.

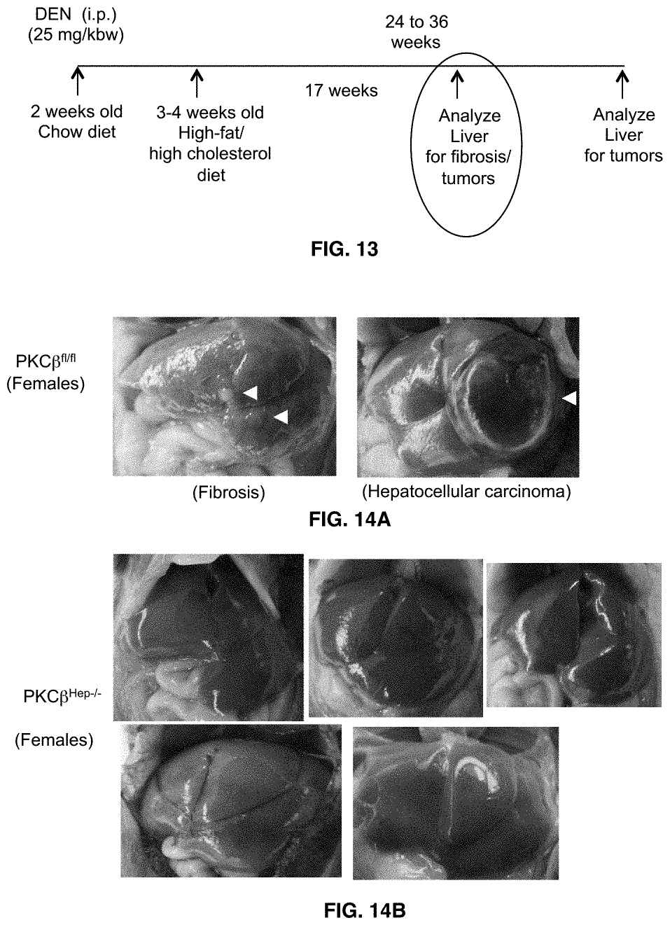

FIG. 13 shows the timeline of DEN-induced HCC analysis. FIGS. 14A and 14B show a comparison of diethylnitrosourea-induced hepatocellular carcinomas between PKCβ^{fl/fl} and PKCβ^{Hep-/-} mice fed a HFHC diet. The data shows that PKCβ^{fl/fl} mice exhibit more liver nodules and HCC relative to PKCβ^{Hep-/-} mice, which is a clear indication that hepatocyte-specific PKCβ deficiency protects from DEN-induced fibrosis and HCC, thus supporting effectiveness of using PKCβ inhibitors in reducing development of nonalcoholic fatty liver disease.

The following references are cited herein:
1. World Health Organization. Obesity: Preventing and Managing the Global Epidemic. Report of a WHO Consultation. World Health Organ Tech Rep Ser 894:i-xii, 1-253, (2000).
2. World Health Organization. Global Health Observatory. Prevalence of Obesity (2016).
3. Bluher M, Nat Rev Endocrinol, 15(5):288-298 (2019).
4. Gregor M F and Hotamisligil G S, Annu Rev Immunol, 29:415-445 (2011).
5. Samuel V T and Shulman G I, Cell Metab, 27:22-41 (2018).
6. Ordovas et al. Curr Opin Lipidol, 22:129-136 (2011).
7. Rankinen et al. Obesity; 14:529-644 (2005) update.
8. Castillo-Armengol et al. EMBO Rep; 20:e47903 (2019).
9. Scheja L and Heeren J J, Hepatol, 64:1176-1186 (2016).
10. Meex R C R and Watt M J, Nat Rev Endocrinol, 13:509-520 (2017).
11. Giudice J and Taylor J M, Curr Opin Pharmacol, 34:49-55 (2017).
12. Fasshauer M and Bluher M, Trends Pharmacol, Sci 36:461-470 (2015).
13. Villarroya et al. Nat Rev Endocrinol, 13:26-35 (2017).
14. Fabbrini et al. Hepatology, 51:679-689 (2010).
15. Mehta D and Mehta K D, Am J Physiol, Liver Physiol. 312:G266-G273 (2017).
16. Bansode et al. J Biol Chem; 283:231-236 (2008).
17. Huang et al. Hepatology, 49:1525-1536 (2009).
18. Huang et al. J Lipid Res, 53:368-378 (2012).
19. Huang W and Mehta K D, Cell Mol. Gastroenterol Hepatol. 1:395-405 (2015).
20. Shu et al. Mol Metab, 44:101133 (2021).
21. Huang et al. J Lipid Res, 45:1519-1527 (2004).
22. Baskin et al. EMBO Molecular Medicine, 6:1610-1621 (2014).
23. Toth et al. Molecular Cellular Biology, 24:8288-8300 (2004).
24. Han et al. Analytical Biochemistry, 330:317-331 (2004).
25. Han et al. Biochemistry, 44:16684-16694 (2005).
26. Huang et al. Journal Biological Chemistry, 286:22795-22805 (2011).
27. Ishizuka et al. Endocrinology Research, 30:287-299 (2004).
28. Kawakami et al. Journal Biological Chemistry, 279:47720-47725 (2004).
29. Bossenmaire et al. Diabetologia, 40:863-866 (1997).
30. Copp et al. Cancer Research, 69:821-1827 (2009).
31. Erion D M. and Shulman G I. Nat Med, 16:400-402 (2010).
32. Lyu et al. Cell Metab, 32:1-11 (2020).
33. Vrecken et al. Biochem Biophys Research Comm, 279:378-382 (2000).
34. Xu et al. Proc Natl Acad Sci USA, 116:11235-11240 (2019).
35. Oemer et al. Cell Report, 30:4281-4291 (2020).
36. Ishii et al. Science, 272:728-731 (1996).

What is claimed is:

1. A protein kinase Cβ inhibitor with a chemical structure 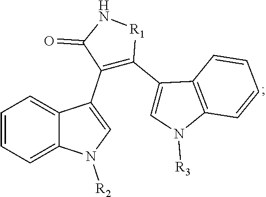
or
a pharmaceutically acceptable salt thereof; wherein,
R₁ is C═O or N; and
R₂ and R₃ independently are H, CH₃, or (CH₂)₄C≡N.

2. The protein kinase Cβ inhibitor of claim 1, wherein the chemical structure is a derivative or analog of bisindolylmaleimide.

3. The protein kinase Cβ inhibitor of claim 1, wherein the chemical structure is 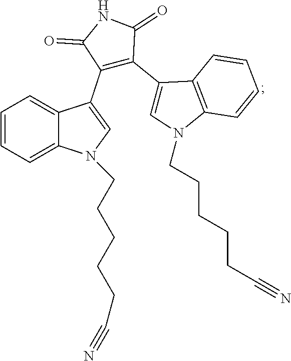 [SMILES: N#CCCCCCn1cc(C2=C(c3cn(CCCCCC#N)c4ccccc34)C(=O)NC2=O)c2ccccc21]
or
a pharmaceutically acceptable salt thereof.

4. The protein kinase Cβ inhibitor of claim 1, wherein the chemical structure is 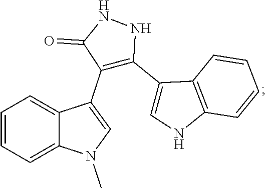 [SMILES: Cn1cc(-c2c(-c3c[nH]c4ccccc34)[nH][nH]c2=O)c2ccccc21]
a pharmaceutically acceptable salt thereof.

5. A pharmaceutical composition comprising at least one protein kinase Cβ inhibitor of claim 1 and a pharmaceutically acceptable carrier.

6. The pharmaceutical composition of claim 5, wherein the at least one protein kinase Cβ further comprises a bisindolylmaleimide analog wherein R₁ and R₂ together with the indolyl nitrogens form a (dimethylamino)methyl-oxa-triazahexacyclic ring or a pharmaceutically acceptable salt thereof.

7. The pharmaceutical composition of claim 6, wherein the bisindolylmaleimide analog has the chemical structure

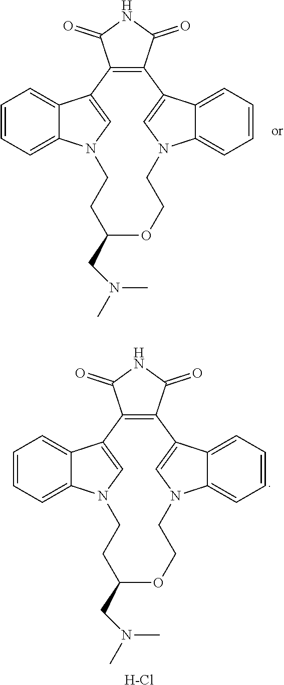

8. A method for treating obesity or an obesity-related disease in a subject in need of such treatment, comprising:
administering to the subject one or more times a therapeutically effective dose of the pharmaceutical composition of claim 5.

9. The method of claim 8, wherein the obesity-related disease is hepatic steatosis, nonalcoholic steatohepatitis or hepatocellular carcinoma.

10. The method of claim 8, wherein the therapeutically effective dose is about 0.5 mg/kg body weight to about 4 mg/kg body weight.

11. The method of claim 8, wherein the therapeutically effective dose is administered daily or every other day.

12. A method for treating a subject suffering from a metabolic disease, comprising the step of:
administering at least once to the subject, in a pharmaceutically acceptable carrier, a therapeutically effective dose of at least one protein kinase Cβ inhibitor with a chemical structure 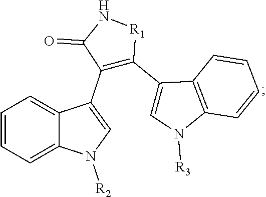
or a pharmaceutically acceptable salt thereof; wherein,
R₁ is C═O or N;
R₂ and R₃ independently are H, CH₃, or (CH₂)₄C≡N or R₁ and R₂ together with the indolyl nitrogens form a (dimethylamino)methyl-oxa-triazahexacyclic ring.

13. The method of claim 12 wherein R₂ is CH₃ and R₃ is H, R₁ and R₂ are each (CH₂)₄C≡N or R₁ and R₂ together form the a (dimethylamino)methyl-oxa-triazahexacyclic ring with the indolyl nitrogens.

14. The method of claim 12, wherein the chemical structure is 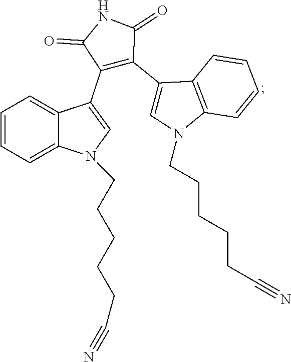 [SMILES: N#CCCCCCn1cc(C2=C(c3cn(CCCCCC#N)c4ccccc34)C(=O)NC2=O)c2ccccc21]
or
a pharmaceutically acceptable salt thereof.

15. The method of claim 12, wherein the chemical structure is 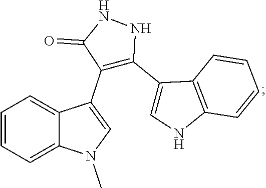 [SMILES: Cn1cc(-c2c(-c3c[nH]c4ccccc34)[nH][nH]c2=O)c2ccccc21]
or
a pharmaceutically acceptable salt thereof.

16. The method of claim 12, wherein the chemical structure is

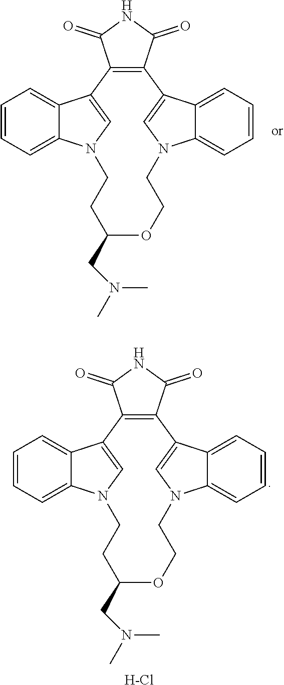

17. The method of claim 12, wherein the metabolic disease is obesity or an obesity-related disease or a combination thereof.

18. The method of claim 17, wherein the obesity-related disease is hepatic steatosis, nonalcoholic steatohepatitis or hepatocellular carcinoma.

19. The method of claim 12, wherein the therapeutically effective dose is about 0.5 mg/kg body weight to about 4 mg/kg body weight.

20. The method of claim 12, wherein the therapeutically effective dose is administered daily or every other day.

21. A method for treating at least one of obesity or other obesity-related disease in a subject in need thereof, comprising the step of:
administering, parenterally, at least once to the subject, in a pharmaceutically acceptable carrier, a therapeutically effective dose of at least one protein kinase Cβ inhibitor with a chemical structure 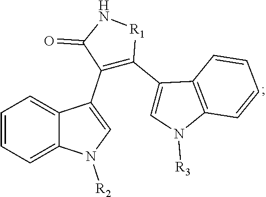
or a pharmaceutically acceptable salt thereof; wherein,
R₁ is C═O or N;
R₂ and R₃ independently are H, CH₃, or (CH₂)₄C≡N or R₁ and R₂ together with the indolyl nitrogens form a (dimethylamino)methyl-oxa-triazahexacyclic ring.

22. The method of claim 21, wherein the protein kinase Cβ inhibitor or a pharmaceutically acceptable salt thereof has the chemical structure

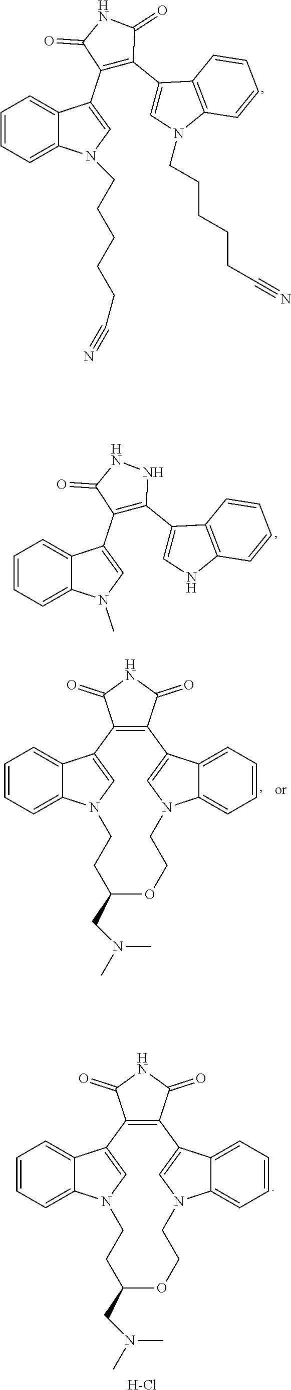

23. The method of claim 21, wherein the step of administering comprises administering the therapeutically effective dose of the at least one protein kinase Cβ inhibitor intraperitoneally.

24. The method of claim 23, wherein the therapeutically effective dose is about 0.5 mg/kg body weight to about 4 mg/kg body weight.

25. The method of claim 21, wherein the step of administering comprises administering the therapeutically effective dose of the at least one protein kinase Cβ inhibitor daily or every other day.

26. The method of claim 21, wherein the obesity-related disease is hepatic steatosis, nonalcoholic steatohepatitis or hepatocellular carcinoma.
